(19) United States
(12) Patent Application Publication
(10) Pub. No.: US 2008/0212032 A1
(43) Pub. Date: Sep. 4, 2008

(54) VISUAL SKILL DIAGNOSTIC AND THERAPEUTIC SYSTEM AND PROCESS

(76) Inventors: Barry L. Seiller, Lake Forest, IL (US); Kathleen S. Puchalski, Mundelein, IL (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/105,029

(22) Filed: Apr. 17, 2008

Related U.S. Application Data
(63) Continuation-in-part of application No. 12/025,881, filed on Feb. 5, 2008.
Application No. 12/025,881 is a continuation of application No. 10/142,360, filed on May 9, 2002, now Pat. No. 7,326,060.

(51) Int. Cl. A61B 3/18; A61B 3/02

(52) U.S. Cl. 351/246; 351/222

(57) ABSTRACT

A system and method for diagnosing a user's visual skills and for therapy is provided. The method disclosed determines the user's visual skill diagnostic score. The visual skill diagnostic score allows the user or the supervising professional to ascertain the user's visual ability. The method is designed to be executed on a computer having a display.

This application is a continuation-in-part of U.S. application Ser. No. 12/025,881 filed on Feb. 5, 2008, which is a continuation of U.S. application Ser. No. 10/142,360 filed on May 9, 2002 (now U.S. Pat. No. 7,326,060) which are incorporated herein by reference in their entirety and upon which priority is claimed.

### TECHNICAL FIELD

The present invention relates to a computer-based diagnostic and therapeutic system and process and, more particularly, to a computer-based diagnostic system and process to determine the visual skills of a user or patient and/or provide treatment.

### BACKGROUND

Various visual diagnostic systems and methods are known. One such example is the Snellen Eye Chart which is used to measure visual acuity. However, more thorough testing and diagnoses is often desirable for some patients, such as those who have recently suffered head trauma and the elderly. While a variety of other diagnosing tests are known, the results are often complex. There is a need for an improved diagnosis system, preferably providing a unified diagnostic score to the user or eye care or other health care doctor, clinician or other professional.

### SUMMARY

The present invention relates to an improved visual skill diagnostic system and process that the claims and only the claims define the invention.

The present process may include the acts of providing a computer system executing a computer program having visual skill evaluation software, conducting at least diagnostic tests where numeric scores may be calculated for each test, and determining a visual skill diagnostic score.

One object of the present invention is to provide an improved visual skill diagnostic method.

### DESCRIPTION OF THE DRAWING FIGURES

FIG. 1 is a flow chart of the various options of the visual skill diagnosis program;

FIGS. 2 and 2a are screen shots of the visual alignment diagnostic test;

FIG. 3 is a flow chart of the visual alignment diagnostic test;

FIGS. 4 and 4a are screen shots of the depth perception diagnostic test;

FIG. 5 is a flow chart of the depth perception diagnostic test;

FIGS. 6 and 6a are screen shots of the visual flexibility diagnostic test;

FIG. 7 is a flow chart of the visual flexibility diagnostic test;

FIGS. 8 and 8a are screen shots of the visual recognition diagnostic test;

FIG. 9 is a flow chart of the visual recognition diagnostic test;

FIGS. 10 and 10a are screen shots of the visual tracking diagnostic test;

FIG. 11 is a flow chart of the visual tracking diagnostic test;

FIG. 12 is a flow chart to determine a visual skill diagnostic score;

FIG. 12a is a flow chart showing an optional weighted use of coefficent.

FIG. 13 is an exemplary illustration of a report produced by the visual skill diagnosis program; and

FIGS. 14a-d illustrate various graphical representations optionally depicting multiple parameters of a visual skill diagnostic score and/or post-training visual skill scores.

FIG. 15 illustrates a physician's prescription order.

FIG. 16 illustrates a progress report document.

FIG. 17 illustrates a progress graph depicting a visual skill diagnostic score and post-training visual skill scores.

### BRIEF DESCRIPTION OF THE PREFERRED EMBODIMENTS

For the purposes of promoting an understanding of the principles, reference will now be made to the embodiments illustrated herein and specific language will be used to describe the same. These are merely examples. It will nevertheless be understood that no limitation of the scope of the invention is thereby intended. Any alterations and further modifications in the described processes, systems or devices, any further applications of the principles of the invention as described herein, are contemplated as would normally occur to one skilled in the art to which the invention relates, now and/or in the future.

As used in the claims and the specification, the following terms have the following definitions:

The term “ocular” means of or relating to the eye, or relating to or using the sense of sight or vision.

The term “machine readable” refers to any information encoded or provided in a form which can be read, scanned or sensed by a computer or computer machine. The machine readable information is capable of being interpreted via hardware, software, or a combination of both.

The term “diagnosis” means to evaluate one or more health or medical conditions.

The term “software” refers any computer program or collection of computer programs that directs the central processor of the computer to perform some tasks on a computer system. The software may be provided on a compact disc (CD), floppy disk, or any other information transferring device or available, downloadable, or executable from a remote source, such as via an internet connection.

The term “computer system” means a computer and/or network of computers, local, via internet, or otherwise and any other software and/or peripheral devices that allow the computer to be functional and operational.

The term “computer” refers to any machine with one or more microprocessors that manipulates data according to a group or set of provided instructions, such as through software.

The term “visual display” means any display or monitor capable of presenting viewable images generated by computer.

The term “visual output” refers to any image, shape, object, character or target presented through the visual display.

The term “linked” refers to the connection of two or more pieces of computer hardware, such as the connection between a computer and a visual display. A laptop computer and its monitor is one example of such linked computer and visual display. The term linked refers to the electrical, physical, wireless, and/or communication connection between two electrical components.

The term “input device” means any piece of hardware to provide information and data to an information processing system such as the computer. An input device may be a keyboard, mouse, joystick, game controller, button, switch and/or sensor of any kind.

The term “visual alignment” refers to the eyes' ability to aim both eyes accurately on a given target. Visual alignment measures where eyes fixate in free space (i.e., exactly on point, in front or behind the target, above or below the target).

The term “depth perception” refers to the ability to see an object in free space and/or judge that object's speed and/or distance. The perception of depth relies on the person's ability to use both eyes simultaneously on a target.

The term “visual flexibility” refers to the skill of moving the eyes efficiently and simultaneously. The term visual flexibility refers to the shift of gaze from near to far and back (binocular skills). Three distinct skills that make up eye flexibility: convergence, divergence, and alternating flexibility. The term “convergence” means the ability of the eyes to maintain an inward posture. The term “divergence” means the ability of the eyes to maintain an outward posture. The term “outward flexibility” means the ability of the eyes to alternate between an inward and outward posture.

The term “visual recognition” refers to how the user remembers stimuli. Well developed visual recognition skills refer to the user's ability to view visual stimuli, process that visual stimuli and respond to the stimuli.

The term “visual tracking” refers to the user's ability to search and scan a field of view as well as locate, process, and react to the items or objects in that field of view during that search process. The term visual tracking relates to the user's ability to track or follow an object.

The term “score” refers to the result of any test or examination. A score can be expressed numerically, alphabetically, graphically, or in any combination thereof or in other form which would depict information to the user.

The term “unified diagnostic score” refers to the unitary or singular result of any test, examination, or series of tests or examinations. A unified diagnostic score can be expressed numerically, graphically, or in any other form which would depict information.

The term “combining” refers to any mathematical operation (typically addition, subtraction, multiplication or division, or a combination thereof) which has as inputs various numbers, scores, or values to create a number, score, or value.

The term “output” refers to any information produced by a computer program and perceived by the user, visually, aurally or otherwise. The output may be produced in tangible and/or intangible form, on screen, printed, email, data record, or otherwise.

The term “therapy” refers to any form of attempted remediation of a health or medical related condition, problem, or ailment.

The term “therapy regimen” refers to any ordered, prescribed, regulated, or directed exercises, training and/or manner of living intended or designed to preserve, restore, improve or attain a health related condition, problem, ailment, or result.

The term “prescription document” refers to any written item or computer output ordering or directing a patient to a therapy regimen. The prescription document may be produced, signed or otherwise authorized by a medical doctor, licensed professional or otherwise authorized individual.

The term “patient identification” refers to any single or group of numbers, letters, characters and/or symbols used to designate a particular person or individual.

The term “computer memory” refers to any computer component, device or recording media capable of retaining digital data for some period of time. Computer memory may refer to the temporary storage of data or the permanent storage of data.

The term “patient trend output” refers to a report, display or representation illustrating a user or patient's scores over a period of time or sequence of sessions. The patient trend report may assist the doctor, eye care professional or end user in evaluating a user's performance or remediation over an extended period of time.

The term “patient's response” refers to an individual's reaction resulting from a given stimulus. A patient's response refers to the user's activation or manipulation of the input device after being presented with computer output, including for example, visual output on the visual display.

The term “memory” refers to an individual's apparent ability to store, retain, and subsequently retrieve information as reflected in a patient's response.

The term “memory image” refers to an image or visual indicia that is presented to the user for a period of time and thereafter removed from the user's view. The user must then recall the image before providing a response via the input device.

The term “amount of time” refers to the time lapse between two given events. An amount of time can correspond to the time between when the user is presented with a memory image and when the memory image is removed from display.

The term “replicate” means to repeat, duplicate or reproduce in whole or in part.

The term “covering” or “covers” refers to the act of placing an object over the user's eye that extends over at least some of the user's field of vision.

The term “lens” is a light transmissive element that covers the eye. It need not magnify or bend light. It may be colored and/or polarized and/or comprise one or more LCD or other screens or image generator located over the eye(s).

The term “color” refers to the visual perception derived from the spectrum of light interacting in the eye with the spectral sensitivities of the eye's light receptors. The colors of the visible light spectrum are red, orange, yellow, green, blue and violet. However, an infinite number of colors can be created through a combination of any or all of the above. Color may include polarization filtering.

The term “coefficient” refers to any constant multiplicative factor or divisors applied to an object, such as a first score or second score. A coefficient may be any real number not equal to one (1).

The term “graphical representation” refers to a graph, chart plot of data or information. The term graphical representation also refers to any pictorial diagram depicting or illustrating the interrelationship of data, variables, shapes, distances, time and/or other parameters.

The term “parameter” refers to any character, aspect, value or element set, established, fixed, varied, measured or tested. A parameter may be accuracy, reaction time, station score, or any other quantifiable characteristic related to a given test.

The term “hand digits” include the thumb, index finger, middle finger, ring finger, and little finger.

The language used in the claims and specification is to only have its plain and ordinary meaning, except as explicitly defined above. Such plain and ordinary meaning is inclusive of all consistent dictionary definitions from the most recently published Webster's dictionaries and Random House dictionaries.

Referring to the figures, a method of diagnosing a medical patient's neurological-muscular status via an ocular interface comprising the acts of optionally executing machine readable visual diagnoses software on a computer; optionally displaying visual output from said visual diagnoses software on a visual display linked with said computer; optionally providing an input device to remit the patient to provide input signaling to the computer in response to said visual display; optionally conducting at least a first diagnostic test and second different diagnostic test with said visual evaluation software running on said computer, said first and second diagnostic test being from the group consisting of: visual alignment test, depth perception test, visual flexibility test, visual recognition test, and visual tracking test; optionally calculating with said computer at least a first score from said first diagnostic test; optionally conduction with said computer at least a second score from second diagnostic test; optionally calculating from said computer a unified diagnostic score based on combining at least a first score and a second score; optionally outputting said unified diagnostic score in a first output.

Optionally the method would further comprise the acts of patient therapy, said therapy optionally comprising the acts of the patient performing at least a first therapy regimen with said visual evaluation software running on said computer; optionally said first therapy regimen being from the group consisting of: visual flexibility test, visual recognition test, and visual tracking test.

Optionally, said first report comprising a physician's prescription document which includes at least patient identification and a therapy prescription.

Optionally, said computer stores in computer memory associated with said patient the results of said first therapy regimen and/or in said the computer outputs said results in a patient trend output.

Optionally, at least one of said diagnostic tests measures the time between an image being displayed to the patient on said display and the patient's response thereto via said input device.

Optionally, at least one of said diagnostic tests measures the accuracy between patient's response and the image displayed to the patient.

Optionally, at least one of said diagnostic test measures the patient's memory by temporarily displaying a memory image to the patient on said display and then removing that image after an amount of time has lapsed. The patient responding via said input device after said lapse to replicate said memory image.

Optionally, at least one of said diagnostic test comprises the acts of covering the patient's left eye with a lens having a first color and covering the patient's right eye with a lens having a second different color and wherein said diagnostic test display on said display at least a first image in said first color and at least a second image in said second color.

Optionally, said scores are numeric and wherein at least one co-efficient is multiplied by at least one of said scores as part of calculating said unified score.

Optionally, said output includes a graphical representation of the said patient's diagnostic testing wherein the graphical representation shows at least two parameters plotted along two respective dimensions.

Optionally, said input device is hand held and may be activated by the patient or health care practitioner with input to the computer from the patient's hand digits, or voice or sound activated, or both hand digits and sound, and preferably as such without requiring a physical movement of the patent's arms or legs.

The system and method provided can be used as a reliable evaluation and training tool that provides a method of diagnosing and improving visual skills. The diagnostic information obtained may serve a role as part of the rehabilitation process in the remediation of visual skills deficiencies. Visual therapy has been a recognized treatment modality for many years. It may be utilized as a non-invasive form of remediation of visual motor disorders.

Typically, an occupational therapist's role is to determine a patient's potential from a thorough evaluation of physical skills and activities of daily living. One of the physical characteristics that are often difficult to assess is the visual system. A patient's visual system plays an important role in how well an individual performs.

In the field of rehabilitation, the goal is to retrain those pre-existing visual skill levels which were deficient due to brain injury or old age. The visual system is made up of a number of components such as visual acuity (eyesight), peripheral vision (field of vision) and visual motor skills. Visual skills may include eye alignment, depth perception, visual recognition (also known as visual memory), visual tracking, convergence and divergence of the eyes, accommodation (focusing), and hand/eye/body coordination. Eye sight, field of vision, and visual skills can all be affected by brain injury. Visual skills affect the patient's function and activities of daily life such as concentration, reading and driving. Limitation in these skills often result in the inability to function at a high level. If the visual input is inaccurate, the result will be a decreased functional activity level. Visual skill deficiencies can also cause undue frustration manifesting itself in behavioral disorders.

According to the program provided, a user is directed to undergo a base line, or diagnostic, assessment of his or her visual skills. Within the diagnostic section of the program, each exercise provided optionally generates a measurement which can be used as the foundation for prescription therapy exercises.

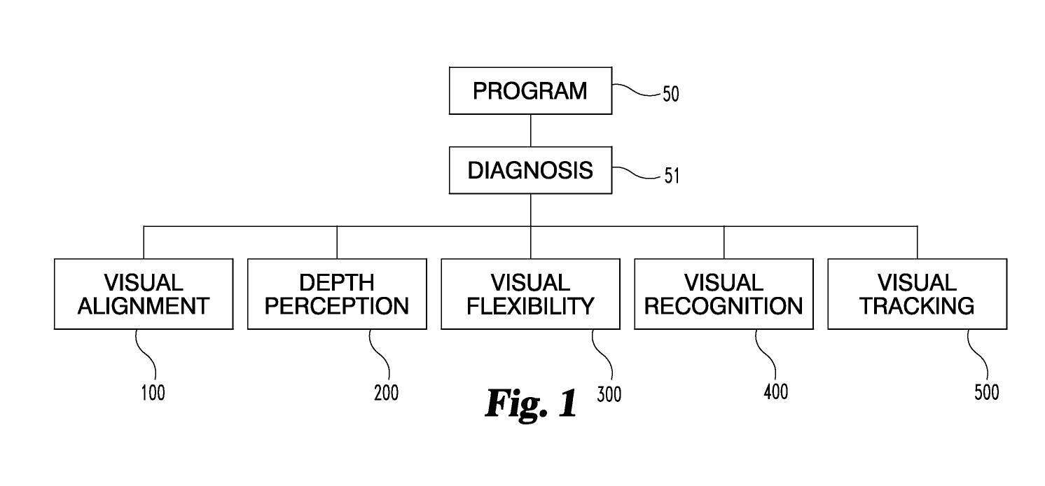

Referring to the flow chart of FIG. 1, the program 50 optionally has a diagnosis menu having a plurality of options for selection. The various options direct the user to various diagnostic tests available. As seen in the figure, the various tests may optionally include: visual alignment 100, depth perception 200, visual flexibility 300, visual recognition 400 and/or visual tracking 500. As will be explained in further detail below, the various diagnostic tests are designed to be interactive programs requiring a user to react to and/or provide input in response to visual indicia appearing on a computer monitor or display. It is also further described below that certain diagnostic tests may optionally test various parameters such as accuracy, timing, complexity, etc.

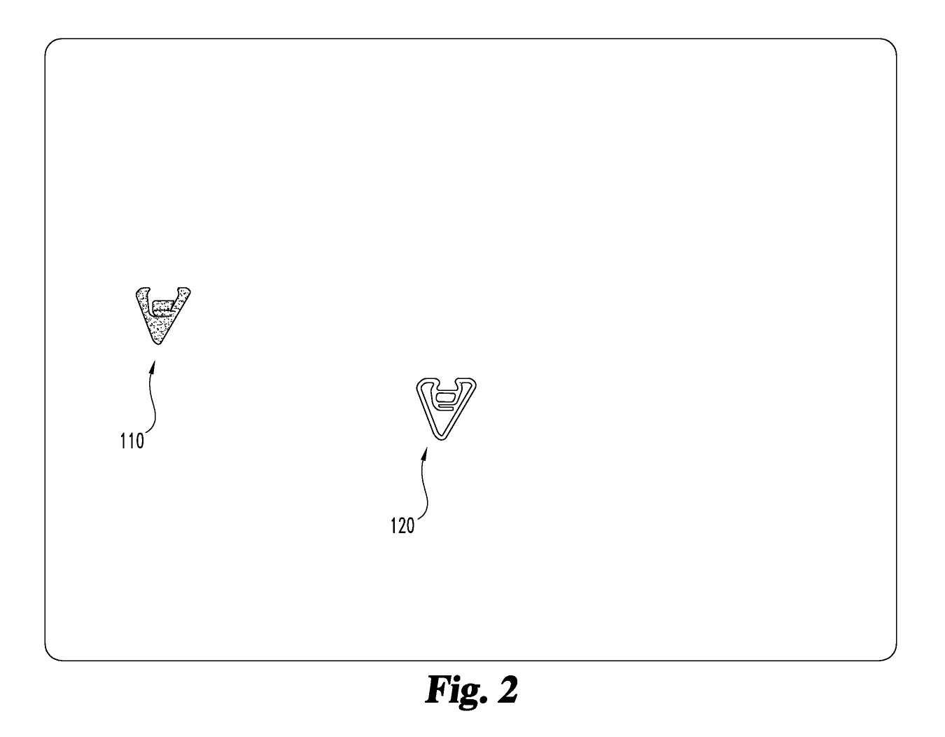

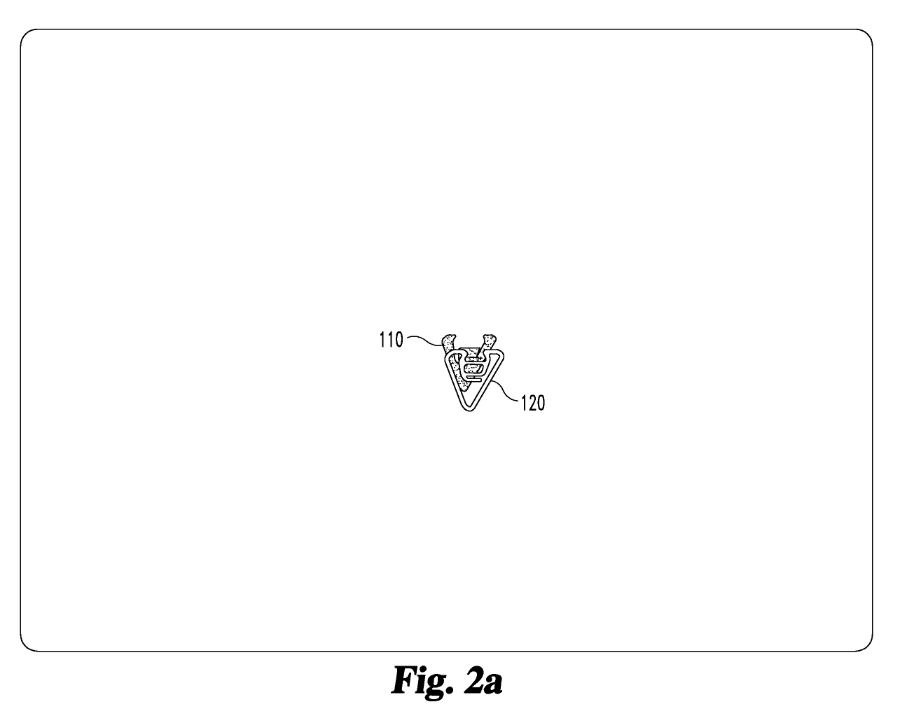

FIGS. 2 and 2a disclose exemplary screen shots displayed during visual alignment diagnostic test 100. Visual alignment diagnostic test 100 determines or measures the user's level of eye alignment. In the preferred embodiment, visual alignment diagnostic test 100 optionally requires the use of glasses to assist in providing the required visual effect. In this regard, the system may optionally include a pair of glasses having a lens of one color (e.g., red) and another lens having a second color (e.g., blue). Optionally, other techniques may be employed to provide a particular visual effect to the user. Alternatively, the glasses may optionally have lenses of different polarity. For example, the left lens may be polarized in a first direction, with the right lens being polarized in a different direction preferably at or about 90° to the first direction. Optionally, one eye may be covered with a polarized lens while the other eye is not. In this embodiment, the pair of glasses optionally has one lens having a horizontal polarity and another lens having a vertical polarity. As shown in FIGS. 2 and 2A, two different objects 110 ands 120 will appear to the user. In the preferred embodiment, one of the objects is optionally red and the other object is optionally blue. While wearing the glasses, the user will manipulate an input device to bring object 110 into alignment with object 120. As shown, object 120 optionally remains stationary while the user manipulates the position of object 110. Once the object appears aligned or overlapped as perceived by the user, the user will indicate as such and the diagnostic test will be complete. Optionally, a combination of different color and different polarity may be used.

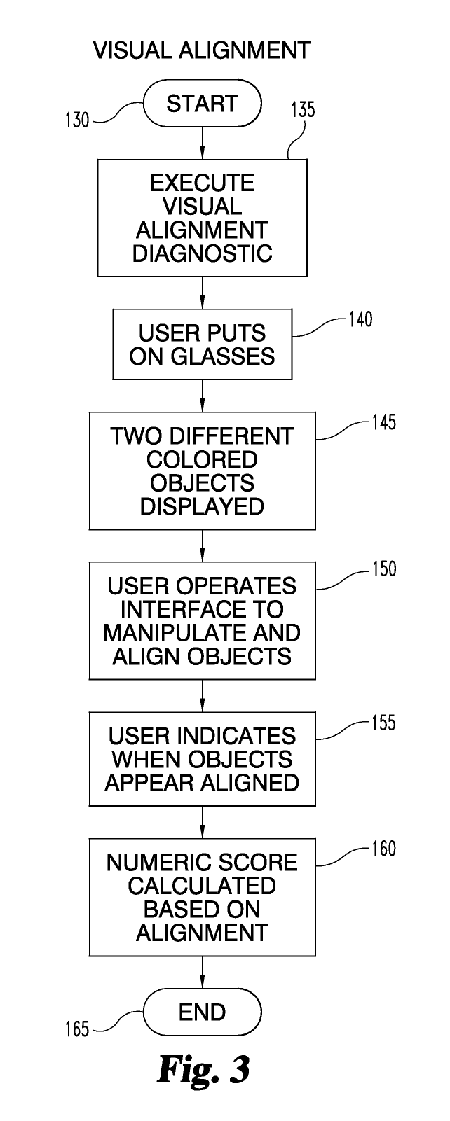

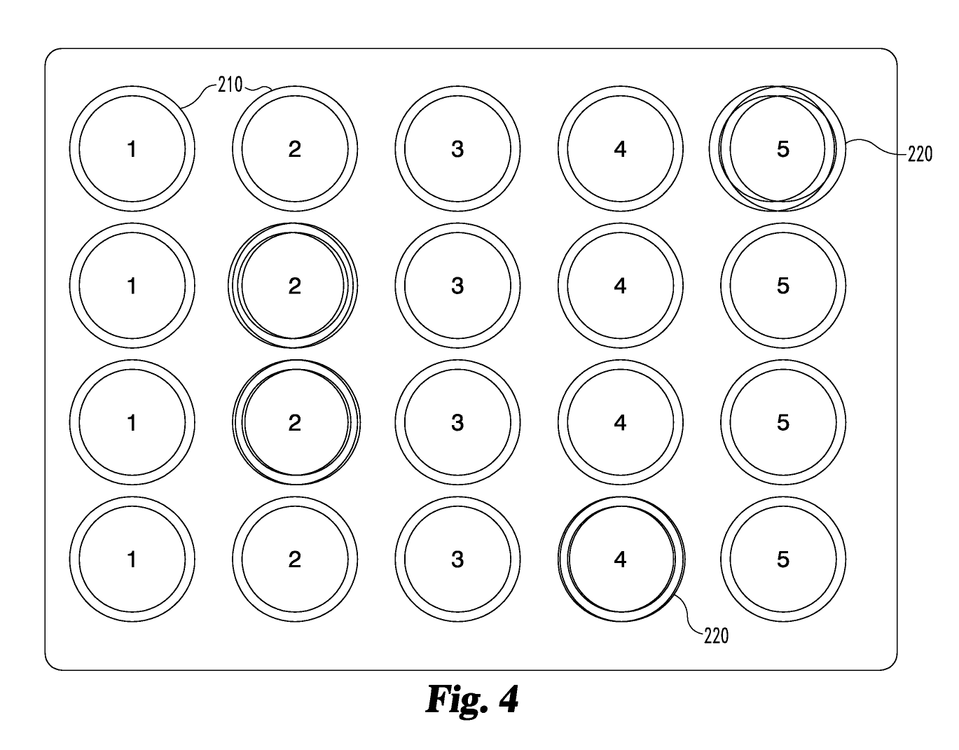

Referring now to FIG. 3, a flow chart is presented depicting the optional methodology employed in visual alignment diagnostic test 100. As shown, the visual alignment program is started (act 130). The user or healthcare professional executes the visual alignment diagnostic test (act 135). To fully assist the user, the user optionally puts on the requisite different-colored lens glasses (act 140). Once the program is up and running, two different colored objects will be presented to the user (act 145). Through the use of user interface, the user then optionally manipulates and aligns the objects in such a way so as to make them appear aligned on the display (act 150). When the objects appears so aligned, the user optionally indicates as such (act 155). Based on the final position, the computer program optionally calculates a numeric score based on the actual alignment versus the alignment determined by the user (act 160). Upon following the completion of the numeric score being calculated, the visual alignment diagnostic test 100 is complete (act 165). Optionally, the sequence of one or more acts in this flow chart and/or the other flowcharts described below may be altered or added to, or occur in parallel or simultaneously. As but one example, the act of donning the glasses 140 may precede the act of starting the program 130.

The numeric score for visual alignment diagnostic test 100 may optionally be based on the horizontal measurement determined. Optionally, the horizontal measurements may range between 0-35, where 0 optionally indicates that the alignment is centered. Optionally, the horizontal measurement measures alignment before and after the center point. Optionally, the visual alignment numeric score is normalized to be consistent with other numeric scores calculated during the diagnostic evaluation. Optionally, the user may indicate a degree of vertical deviation. Hypertropia and hypotropia may be determined if the images appear vertically displaced. In one embodiment, the numeric score determined is independent of the vertical displacement indicated by the user.

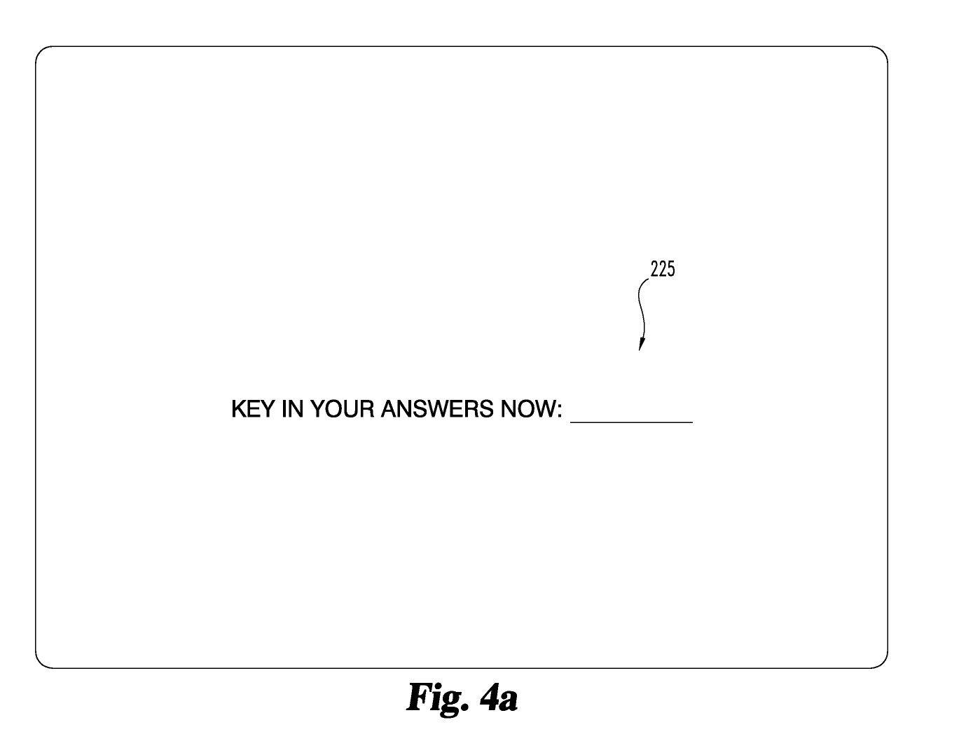

Referring now to FIGS. 4 and 4a, exemplary screen shots of depth perception diagnostic test 200 are shown. Similar to visual alignment diagnostic test 100, depth perception diagnostic test 200 optionally requires the use of special different colored lens glasses. As shown in FIG. 4, various rows and columns of circles 210 are optionally displayed to the user. As shown, each circle in each row may be optionally numbered, or identified in a certain way. Optionally, one circle 220 in each row will appear to float on or off of the screen optionally following a short timetable. Optionally, the user is then prompted 225 to input the number of the circle the user perceived as floating. Preferably, the depth perception testing become progressively more difficult. This allows for gradations of scoring. The preferred example here is that the degree of depth perception separation becomes less with each row (e.g. top to bottom, or otherwise) (see FIG. 4).

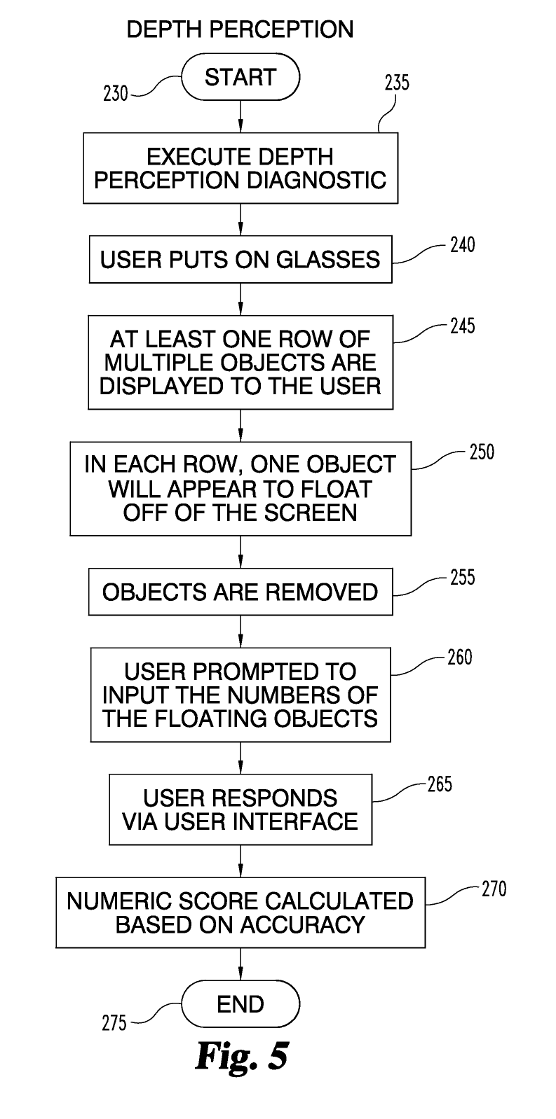

Referring now to FIG. 5, a flow chart is depicted showing the optional methodology of depth perception diagnostic test 200. The program is started (act 230), wherein the user or the healthcare professional optionally executes the depth perception diagnostic test (act 235). The user optionally places the requisite different lens glasses on to assist the user in perceiving the three-dimensional objects (act 240). At least one row of multiple objects is then presented to the user (act 240). In the preferred embodiment, the objects may optionally be circles; however, various other shapes are considered, such as triangles or squares. In each row, one object will optionally appear to float off the screen, such as to have depth in a third-dimension (act 250). Optionally, after a pre-determined amount of time, the rows and columns of objects are removed (act 255). The user is prompted to input the number, or other identifying indicia, of the floating objects (act 260). The user optionally responds through the user interface (act 265). Based on the accuracy of the user's response a numeric score is calculated (act 270), thus concluding the depth perception diagnostic test (act 275).

The numeric score for depth perception diagnostic test 200 may optionally be based on the number of floating objects 220 correctly identified. Though four rows are illustrated, any number of rows having any number of columns may optionally be presented to the user. After the test is completed, the numeric score may optionally be calculated by dividing the number of correctly identified floating objects by the total number of rows presented to the user. Optionally, the depth perception numeric score is normalized to be consistent with other numeric scores calculated during diagnostic evaluation.

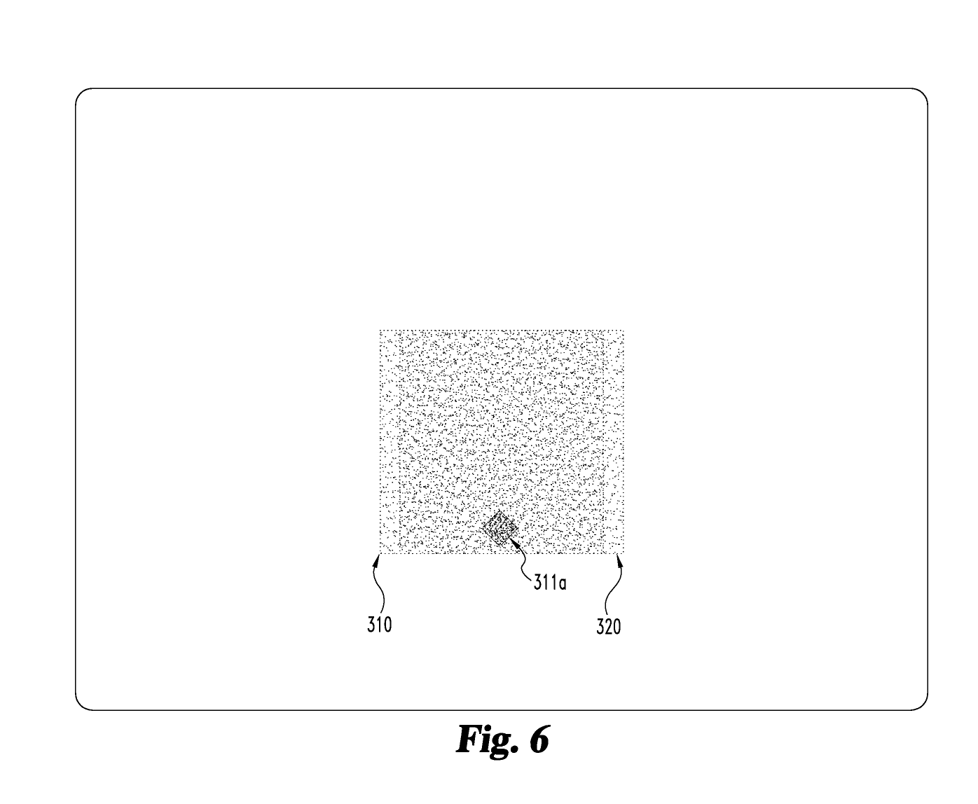

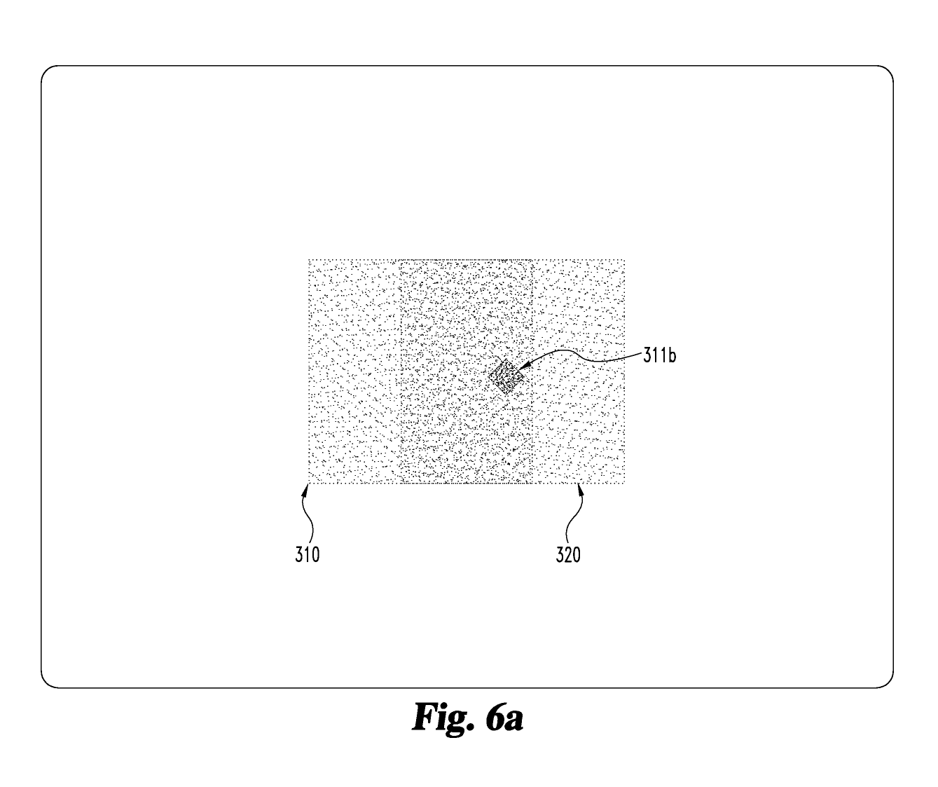

Referring now to FIGS. 6 and 6a, exemplary screen shots of visual flexibility diagnostic test 300 are shown. The visual flexibility diagnostic test 300 provides a base line score for the user's convergence and divergence. Once the user is within visual flexibility diagnostic test 300, the patient will optionally view two super-imposed dotted boxes 310 and 320. Within the boxes, the user should optionally perceive a three-dimensional shape, such as a diamond 311a, 311b, that appears is the overlapping portions of boxes 310 and 320. The shape may optionally appear at the top, bottom (see FIG. 6, shape 311a), left or right (see FIG. 6a, shape 311b) of the overlap of boxes 310 and 320. After the user has indicated the location of the three-dimensional object, the program will progress in difficulty by separating the two original boxes 310 and 320, thereby making it harder to see the three-dimensional target. Optionally, any shape, character, object or visual image may be used other than the diamond example. Optionally, boxes 310 and 320 are different colors. FIG. 6 shows the boxes in a relatively low level of difficulty, whereas FIG. 6a shows it with an increased level of difficulty.

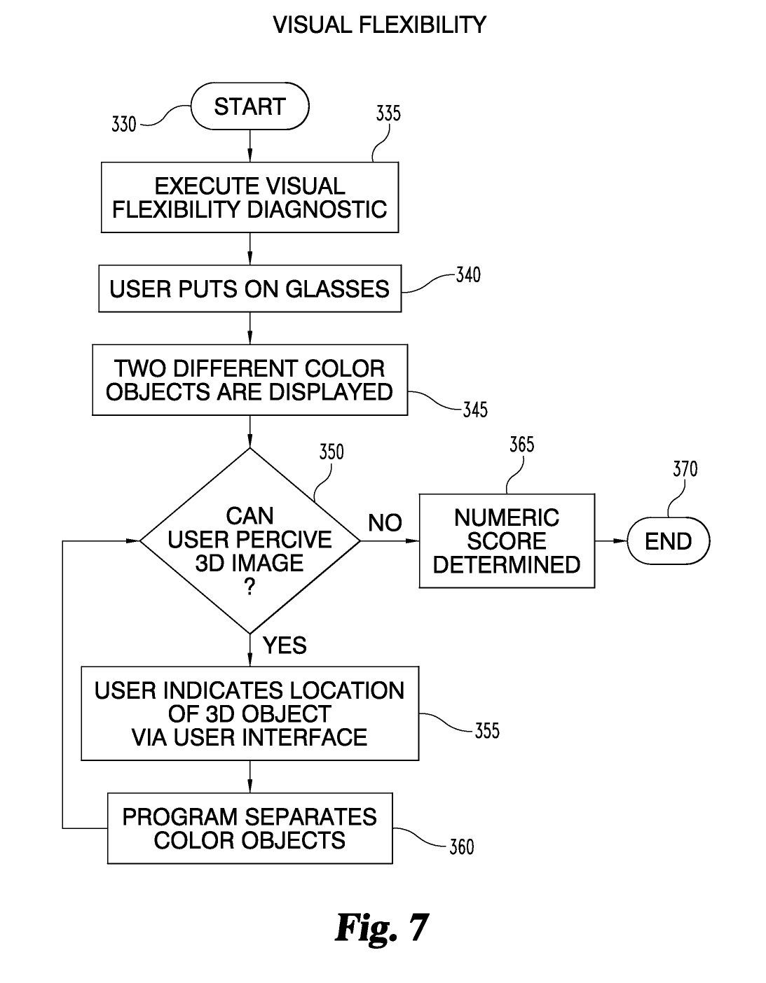

Referring now to FIG. 7, the methodology of visual flexibility diagnostic test 300 is depicted. As shown, the program is started (act 330), wherein the visual flexibility diagnostic test is executed (act 335). Again in this diagnostic test, the user optionally wears the different colored lens glasses (act 340) described hereinabove. In one embodiment, two different colored objects are displayed to the user. Optionally, one red and one blue dotted box appear as super-imposed, or overlapped, on top of one another (act 345). At this point, the user optionally determines if a three-dimensional object is perceived (act 350). The user will optionally indicate the location of the three-dimensional object via the user interface (act 355). Because the user was able to perceive the three-dimensional object, the program optionally separates the colored boxes (act 360). The scoring of this is optionally referred to as a station score. This process iterates until the user can no longer perceive the three-dimensional image. At that point, the numeric score is determined based upon the amount of separation, and/or the user's accuracy and time taken to perceive the three-dimensional object after the boxes have been separated (act 365). At that point, the visual flexibility diagnostic test is complete (act 370).

The numeric score for visual flexibility diagnostic test 300 may optionally be based on the time, accuracy, and/or station score. The test optionally measures the time between the separation of targets 310 and 320 and the user's indication of the location of the three-dimensional target. The accuracy parameter corresponds to the user's correct identification of the location of the object within the overlapping area. The station score optionally represents a measurement of the maximum amount of separation of objects 310 and 320 achieved during visual flexibility diagnostic test 300. Optionally, the numeric score may be determined by considering the percent correct of input responses and the achieved score of the maximum possible station score. Optionally, the numeric score may also be dependent on the speed in which the user responds. Optionally, the visual flexibility numeric score is normalized to be consistent with other numeric scores calculated during diagnostic evaluation.

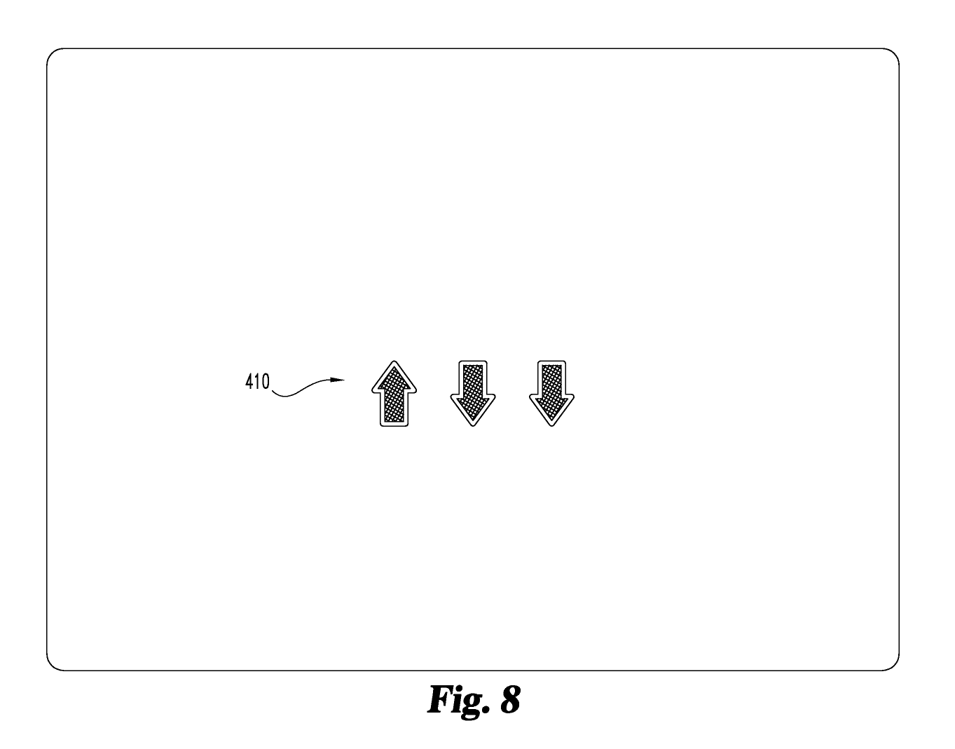

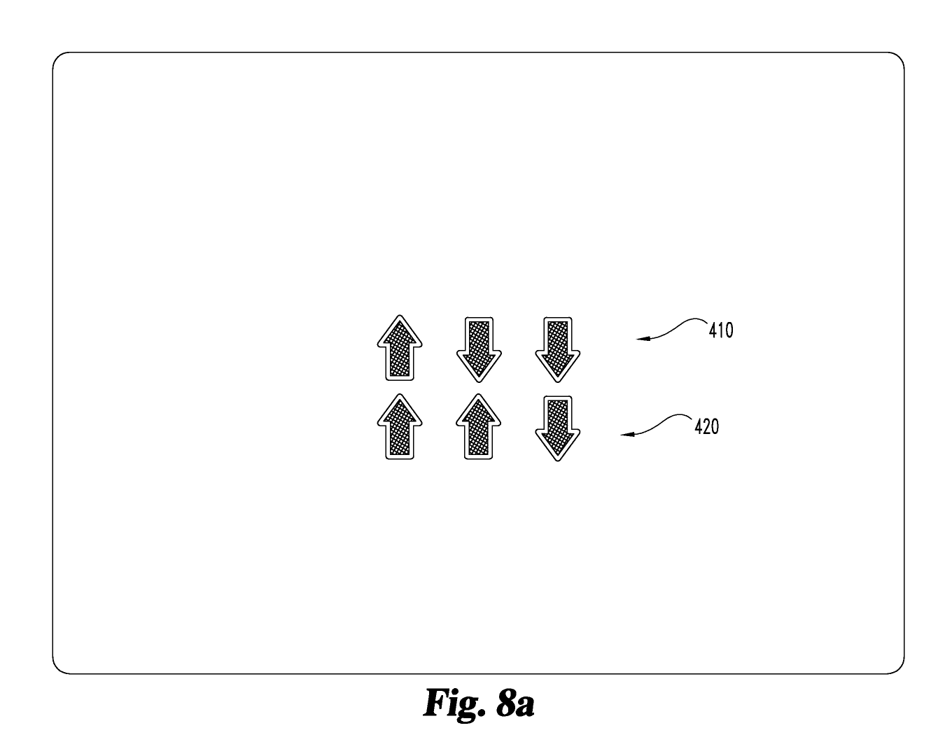

Referring now to FIGS. 8 and 8a, exemplary screen shots of visual recognition diagnostic test 400 are shown. The purpose of visual recognition diagnostic test 400 is to have the user optionally view a series of arrows pointing in various directions. These arrows will flash and disappear on the screen. Once the arrows, or other visual indicia, disappear, the user optionally determines the direction that each arrow pointed, and then repeat it in order using the user interface. Within this diagnostic test, the user is to replicate the series of arrows by indicating the correct direction each arrow points. Therefore, as shown in FIG. 8, in this case a group of arrows 410 are optionally displayed to the user. After a pre-determined time, the visual indicia is optionally removed, wherein the user then optionally recalls the direction of this series. As shown in FIG. 8a, as the user indicates, from memory, the direction of the arrow in the series 420. The program will also display the corresponding previously displayed arrow 410 above the user's response 420.

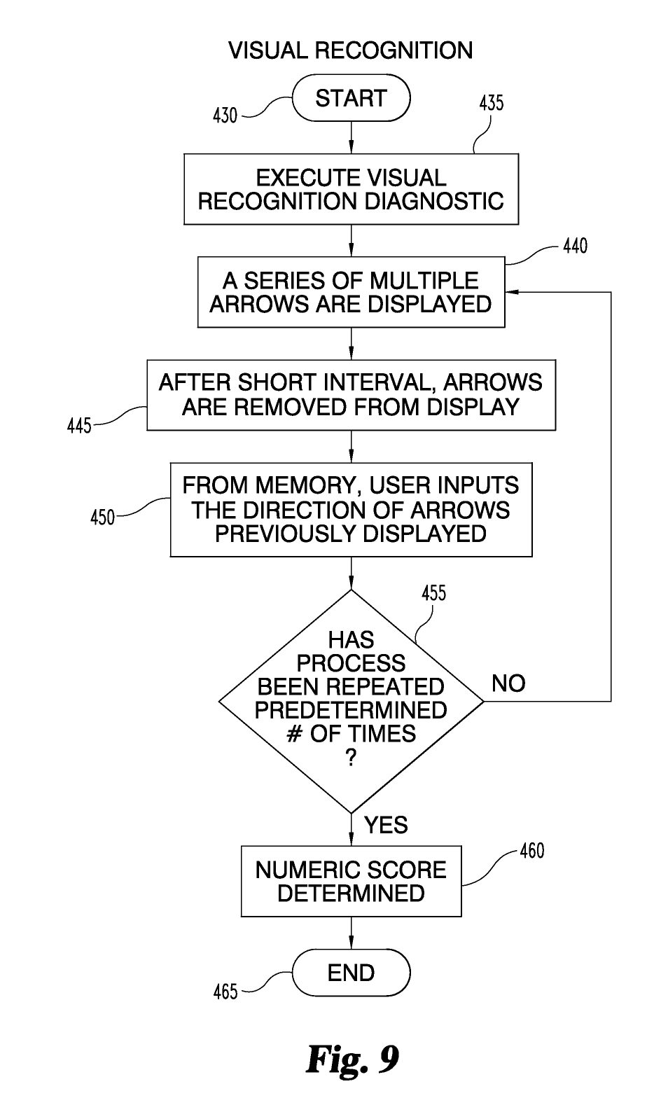

Referring now to FIG. 9, the methodology of visual recognition diagnostic test 400 is shown. Optionally, the program is started (act 430), and the user or healthcare professional optionally executes visual recognition diagnostic test (act 435). As the diagnostic test is executed, a series of multiple arrows are displayed (act 440). After a short interval, the arrows are removed from the display (act 445). Thereafter, from memory, the user optionally inputs the direction of arrows previously displayed via the user interface (act 450). This process optionally repeats a requisite number of times (act 455). If the user has not completed the test, the process will repeat from act 440. If the process has been repeated the requisite number of times, the numeric score for the visual recognition diagnostic test will optionally then be determined (act 460). At which time, the visual recognition diagnostic test is complete (act 465).

The numeric score for visual recognition diagnostic test 400 may optionally be based on the time and accuracy of the user's response. Accuracy is optionally measured as the percent of user responses that are correct. Time is optionally measured as the user's reaction time between when the row of images is removed from display and when the user responds accordingly. Optionally, the slowest reaction time capable of being recorded is 10 seconds, whereas the fastest time optionally recorded is 0.1 seconds. Optionally, the visual recognition numeric score is normalized to be consistent with other numeric scores calculated during diagnostic evaluation.

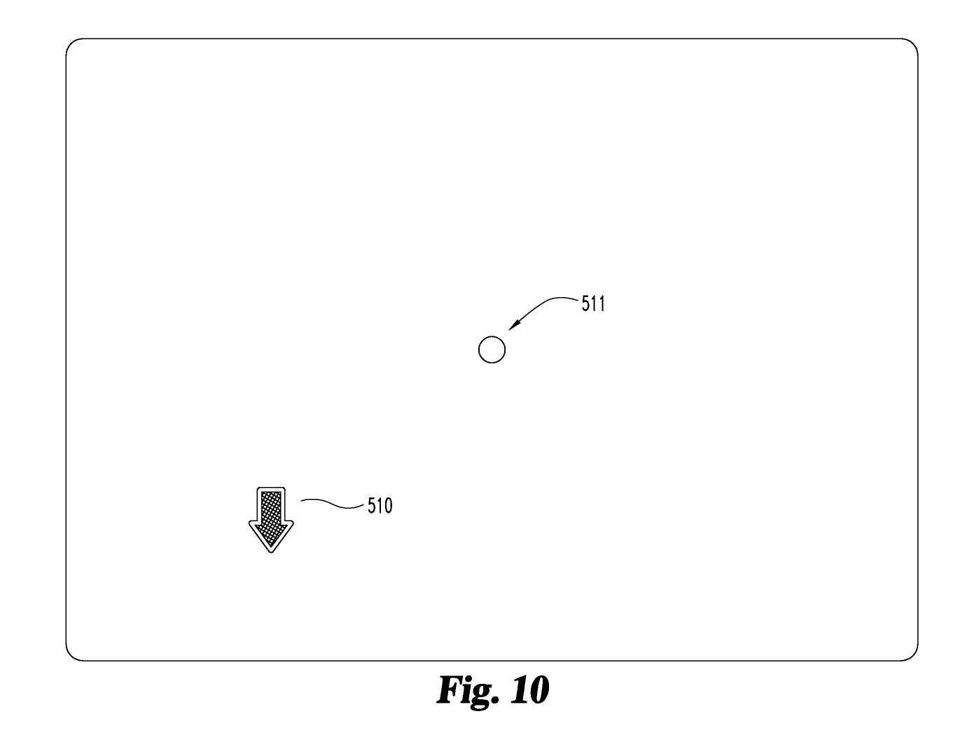

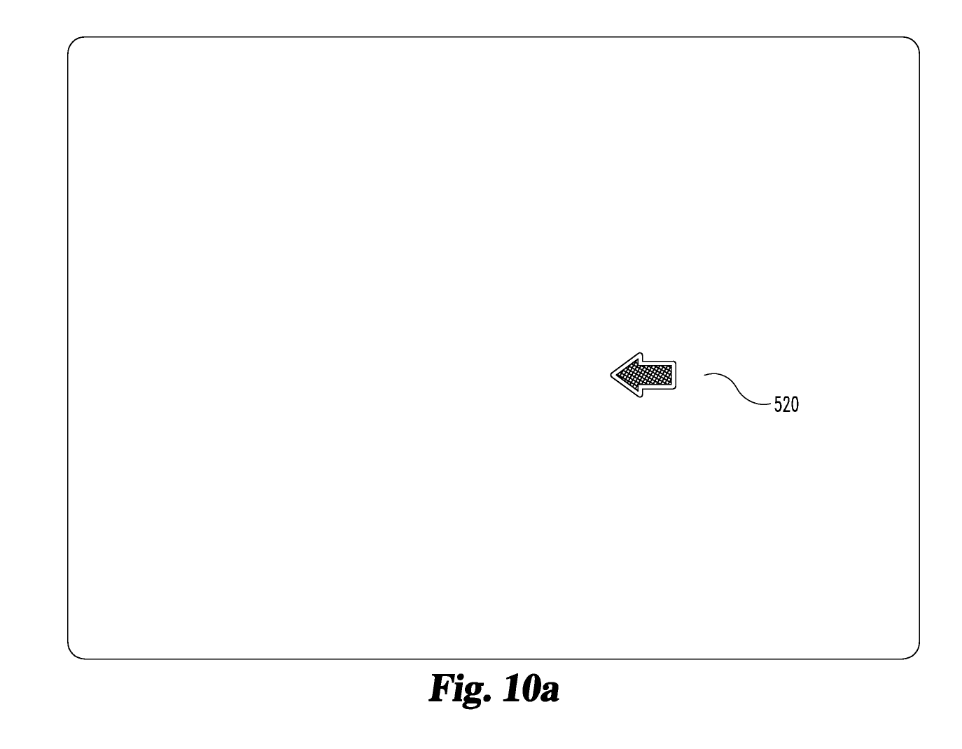

Referring now to FIGS. 10 and 10a, exemplary screen shots of visual tracking diagnostic test 500 are shown. As shown in FIG. 10, the program optionally presents a target, in this case an arrow 510, pointing in a particular direction to the user. After a short period of time, the arrow 510 is removed, where the user then optionally replicates the arrow by indicating the direction of the arrow. Upon input or response by the user, a further target, or arrow 520, is optionally presented to the user at a different location and in a different direction. Another optional feature is shown as object 511 shown in FIG. 10. This is an example of a fixation image. Preferably it appears at or near the center of the screen. It may, by contrast, be omitted (see FIG. 10a without a fixation image). The fixation image is a spot that the user visually focuses on during a test or an exercise (more typically during a therapeutic exercise), whilst the images (such as arrow 510) appear and disappear on the periphery. This allows work on peripheral vision. Optionally, the fixation image can be turned on, off, or in another mode, such a with a set-up or control screen or button. One such other mode could include random appearance and disappearance of the fixation object 511. One optional use of this is to have the computer program set up to only score correct answers (and optionally to penalize any answer) when an answer is given while there is no fixation object appearing on the screen.

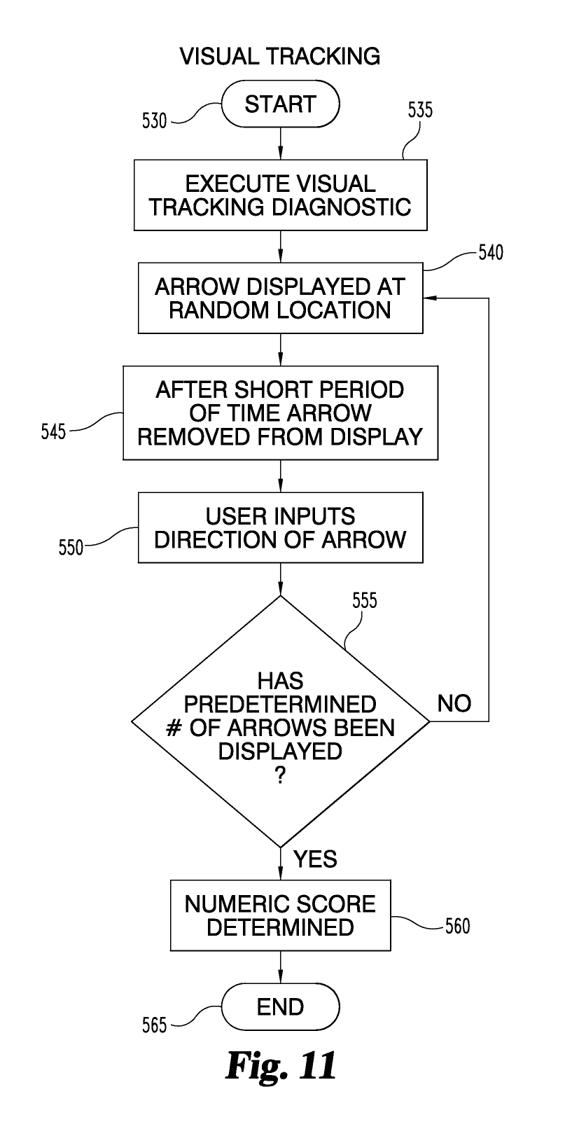

Referring now to FIG. 11, the methodology of visual tracking diagnostic test 500 is shown. The program is started (act 530) and the user or healthcare professional executes visual tracking diagnostic test (act 535). Optionally, an arrow, or other visual target, is displayed at a random location and a random direction (act 540). After a short interval, such as a tenth of a second or three tenths of a second, the arrow is optionally removed from display (act 545). Therein, the user is encouraged to quickly input the direction of the previously displayed arrow (act 550). At this point, this process may optionally continue until the user has responded to a pre-determined number of arrows (act 555). Once the user has responded to the requisite number of arrows, the numeric score will be determined (act 560) and thereafter that test is complete (act 565).

The numeric score for visual tracking diagnostic test 500 may optionally be based on the time and accuracy of the user's response. Optionally, the results are recorded as the percentage correct and average reaction time. Reaction time is optionally measured as the time between the presentation of the image and the entry of the user's response. Optionally, the slowest reaction time capable of being recorded is 10 seconds, whereas the fastest time optionally recorded is 0.1 seconds. Optionally, the visual tracking numeric score is normalized to be consistent with other numeric scores calculated during diagnostic evaluation.

Optionally, any or all of the features may be sped up or slowed down, varied in size, shape, multiplicity and/or type. Typically, this is done on the therapeutic regimens, whilst preferably maintaining the diagnostic parameters constant for consistency/comparability of diagnostic results and data. Hence, preferably the diagnostic regimens are set on default levels. For example, with respect to therapeutics the speed of presentation of the visual tracking routines, visual tracking routines, any time based routine, may be adjusted by the user and/or the therapist. Preferably, this optional feature is controlled by one or more computer screens associated with system set-up, user log in or otherwise. Preferably, speed setting(s) are (optionally) maintained in computer memory on a user basis, and/or on a user session basis, and may be automatically invoked by log-on by that particular user number in subsequent sessions. They may also be kept in memory for tracking and output purposes, and may be combined or factored into (by coefficient or otherwise) scoring, including a unified diagnostic score. Likewise and with similar variables and controls discussed above regarding adjusting speed, optionally the size of the visual output (including for example a memory image or otherwise) may be adjusted, as well as the number of objects, and otherwise. Thus, for example, objects may be made larger so they are easier to see by a patient better served by this adjustment.

Optionally, the diagnostic numeric scores may be directly proportional to user accuracy. Optionally, the numeric scores may be inversely proportional to time and reaction time. With regard to visual flexibility diagnostic test 300, the numeric score may optionally be directly proportional to the station score. Optionally, the numeric scores may be dependent on the percentile percentages correlated to a given parameter measured.

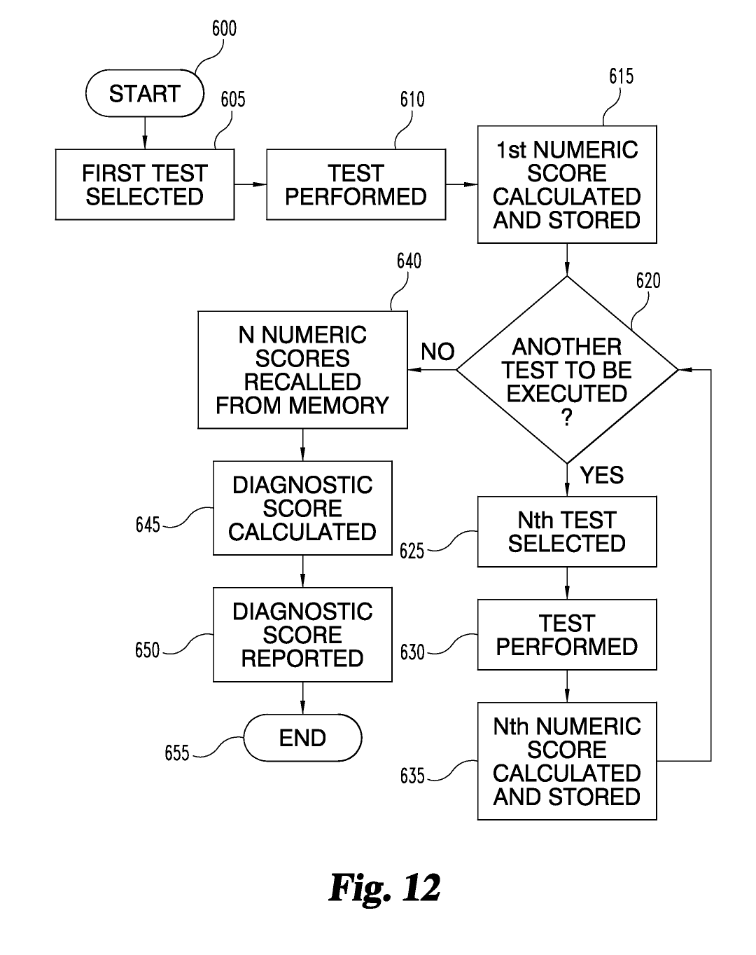

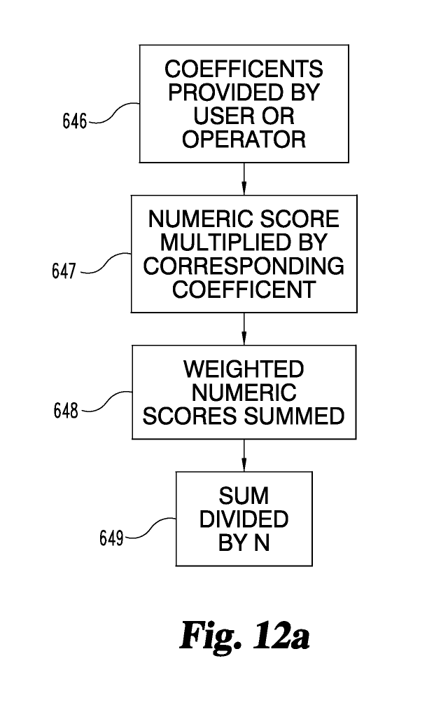

FIG. 12 illustrates a flow chart wherein a single diagnostic score is determined and reported based on an execution and calculation scores based on a number of diagnostic tests. As shown, the program is started (act 600). The user or healthcare professional optionally selects the first diagnostic test to be performed (act 605). The user, either independently or with the assistance of the healthcare professional, will execute and perform that test (act 610). At that point, a first numeric score is optionally calculated and optionally stored (act 615). It is optional that all five visual tests are to be performed. However, it is preferable that more than one diagnostic test is performed. Therefore, if another test is to be executed it is (act 620), the second or third test is selected (act 625) and that test is then performed as well (act 630) and that score is then calculated and stored as well (act 635). When no further tests are to be performed, the multiple numeric scores from each diagnostic test are optionally recalled from computer memory (act 645). With these given tests, a single diagnostic score is optionally calculated or determined (act 645) and this score is then optionally reported to the user or healthcare professional (act 650). At this point, the diagnoses method is complete (act 655).

It is also optional that a user or healthcare professional can weigh the different diagnostic tests. Optionally, program 50 will have predetermined coefficients assigned to particular diagnostic tests. Alternatively, the user or healthcare professional optionally provides various coefficients to the different tests. These coefficients either cause a particular diagnostic test to weigh more heavily in the diagnostic score or to have a less of a bearing on the diagnostic score. Optionally, each numeric score is be multiplied by its corresponding coefficient. Optionally, as but two examples of these weighted numeric scores may summed, or summed and divided by the total number of tests actually performed, resulting in a unified diagnostic score.

Optionally, referring to FIG. 12a, coefficents may be provided by user or optionally by an operator at act 646. Thereafter, optionally the numeric score may be multiplied (or other mathematical operator such as division or otherwise) by other corresponding coefficient at act 647. These may be combined in any number of mathematical operations, preferably by addition such as weighted numeric scores being summed at act 648. Optionally, those sums are divided by the number N corresponding the number of tests, thereby averaging the weighted scores at act 649. Optionally, the foregoing may be done without waiting, but by a simple averaging without coefficents.

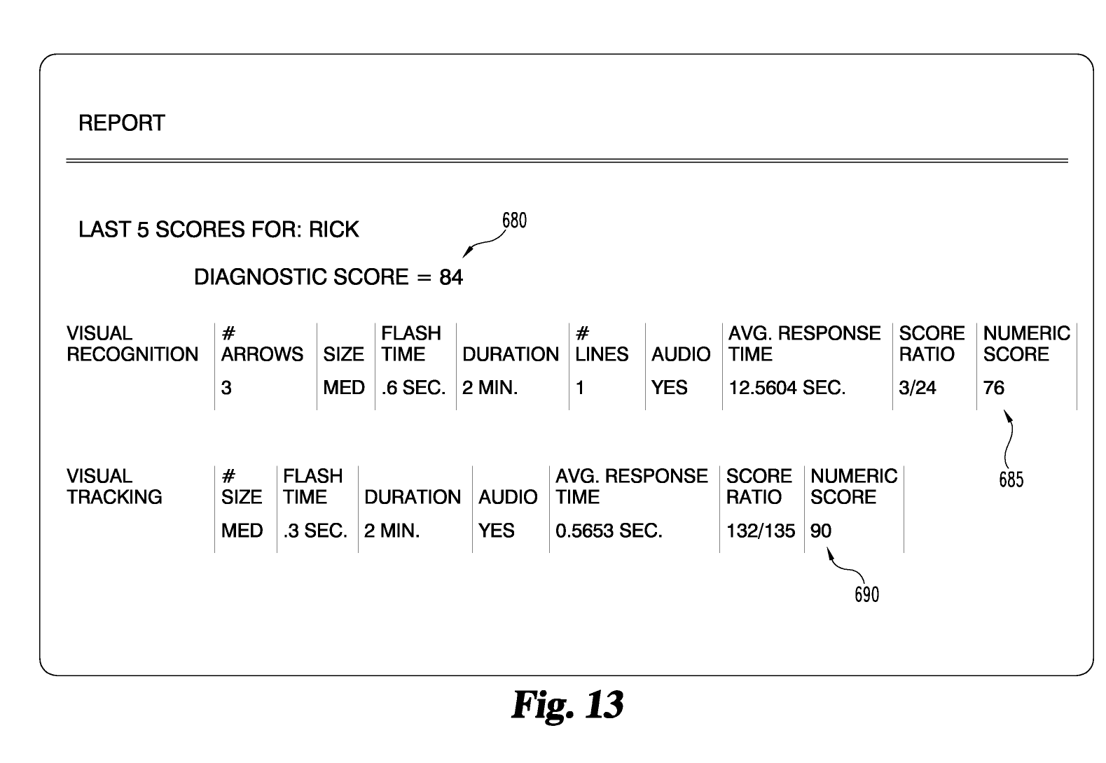

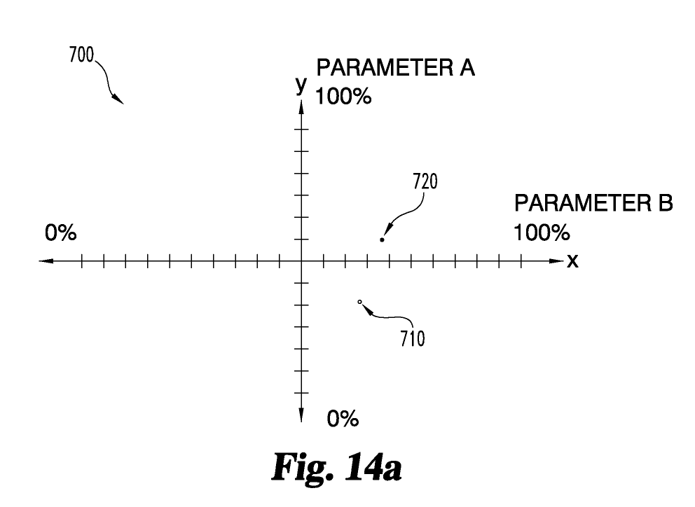

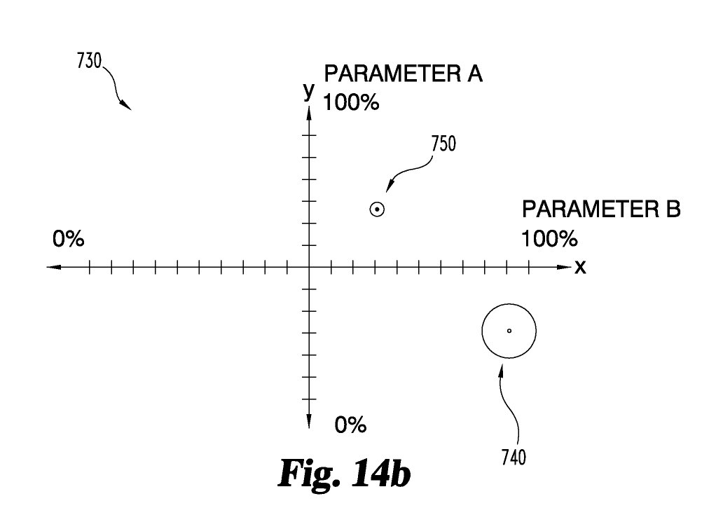

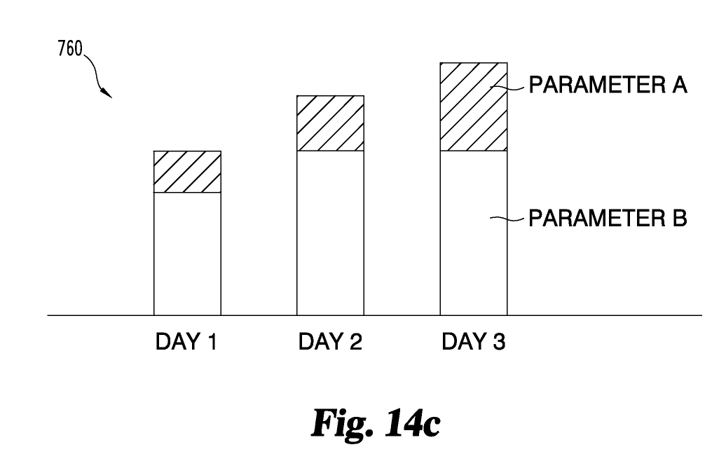

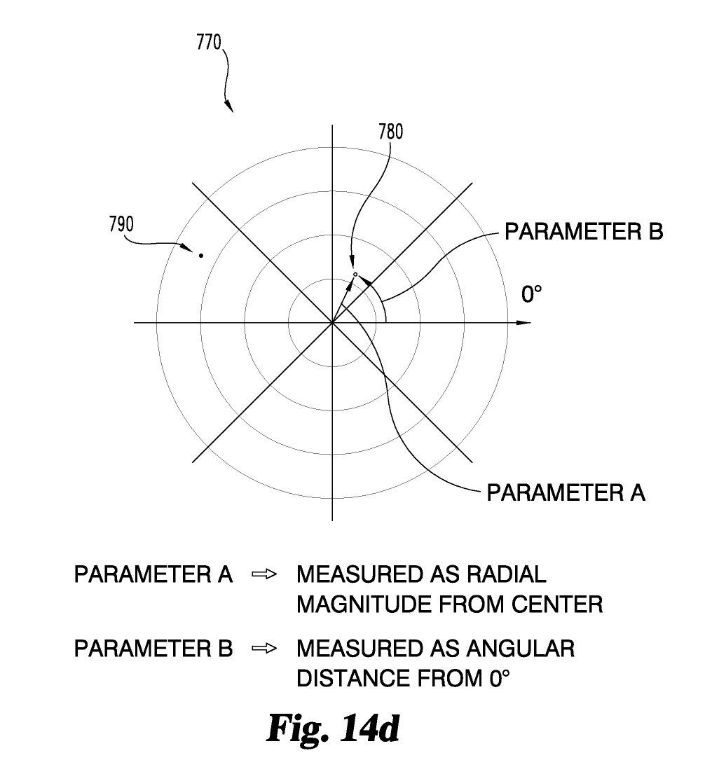

Referring now to FIG. 13, one example of a written report is illustrated. In this example, the written report optionally displays the combined diagnostic score 680 that was the result of two diagnostic tests. As shown, visual recognition diagnostic test 400 was performed, as well visual tracking diagnostic test 500 was performed. The numeric score for visual recognition diagnostic test 400 is optionally displayed 685, as well as the numeric score 690 of visual tracking diagnostic test 500. Other representations of the numeric scores and/or diagnostic score are contemplated. Examples are shown in FIG. 14a-d.

FIG. 14a shows a possible graphical representation of Parameter A and Parameter B of a numeric score or unified diagnostic score. Graphical representation 700 shows both the diagnostic score 710, as well as a post-training score 720. Such a graphical representation 700 will allow the user or healthcare professional to quickly verify that the user has made substantial progress in time and has made slight progress in accuracy.

FIG. 14b depicts a bubble graph. Graph 730 represents Parameter A along the Y-axis and Parameter B along the X-axis. Graph 730 shows unified diagnostic score 740 and post-training score 750. As used herein, a bubble graph is a two-dimensional plot where a third parameter is represented by the size of the points or the area of the circles surrounding the point.

FIG. 14c graphically displays a bar chart illustrating a patient's trend output. As shown, bar chart 760 graphically displays two parameters, Parameter A and Parameter B, for multiple days. In this case, a physician may quickly ascertain the user or patient's trend output over a particular time period related to two parameters. Optionally, bar chart 760 may depict one or more parameters.

FIG. 14d illustrates polar coordinate graph 770. Graph 770 displays both unified diagnostic score 780 and post-training score 790. In this embodiment, Parameter A is optionally measured as a given point's radial distance or magnitude from the center of graph 770. Optionally, Parameter B is measure as the angular distance from 0°, or the positive X-axis.

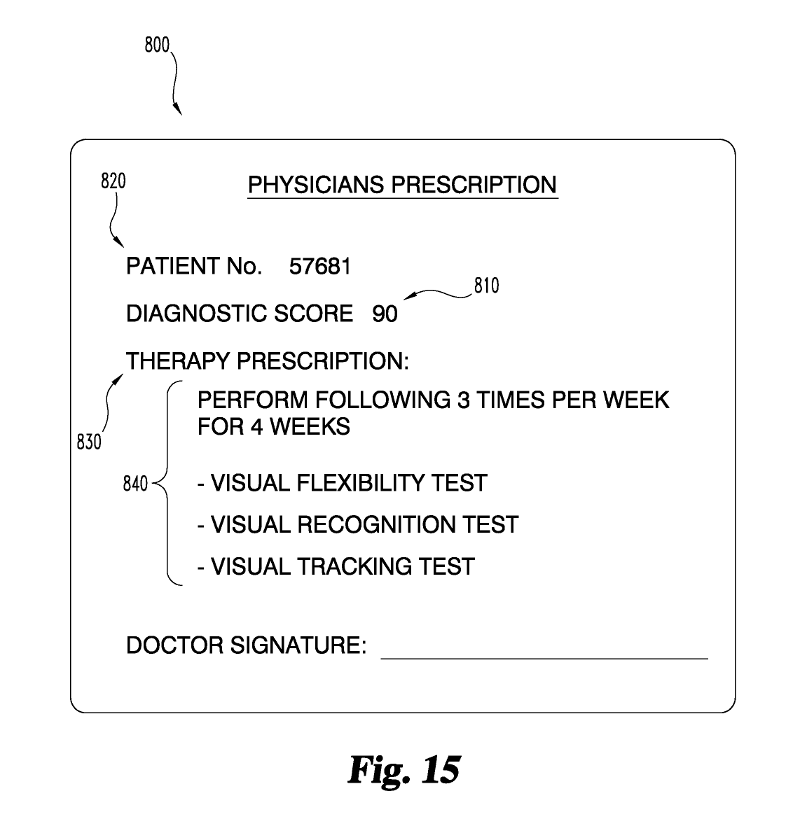

FIG. 15 is physician's prescription document 800. Prescription document 800 is but one example of the possible outputs of the disclosed method wherein the unified diagnostic 810 score is outputted. Optionally, the prescription document 800 includes a patient number 820 and a therapy prescription 830. Optionally, the therapy prescription 830 includes a therapy regimen 840 prepared or designed for the particular patient.

The provided software may optionally be available and/or executable from a remote source. Additionally, the computer utilized by the user/patient may be connected to a remote database, optionally connected via an internet connection. This remote database may optionally maintain patient identification numbers, diagnostic scores, therapy information, and/or other medical information. This database may optionally be accessed via an internet connection.

Further, the user may optionally utilize a web-site based scoring system. In such a system, the user/patient optionally logs in by entering the requisite identifying information, such as a user name and associated password. Once logged in, the user may optionally choose from the diagnostic and therapeutic tests described hereinabove. In this embodiment, the software may be maintained separately from the user's computer and executable from a remote source. Alternatively, the software may be executed on the user's computer, while the testing information and results may be communicated to the web-based scoring system. The results of the tests or exercises may optionally be displayed to the user. Optionally, the particular scores, as well as the date and time of when the test or exercise was conducted, may be stored in the remote database. The stored information may optionally be accessed by the user or by the user's physician or supervising healthcare professional.

Additional information may also be maintained in the remote database. For example, the user or physician or supervising healthcare professional may optionally enter medical information related to the particular user. The medical information entered may also include the particular type of head injury or trauma suffered by the user. A vision survey may also be administered to assist the physician in diagnosing the user's level of visual impairment. The results of the vision survey may optionally be entered and stored into the remote (or a local) database. The collection of various forms of information may allow for future trend-spotting and/or cross-correlation and/or other analysis to be performed. As the amount of information stored in the remote database increases, a physician is able to correlate certain visual skill characteristics and diagnostic scores to particular head injuries or traumas. Further, percentile rankings may be determined, allowing the user or physician to gauge the user's progress relative to other user's of the system. For example, the present invention may be used in connection with a database. Such database may optionally include a variety of fields, including patient identification, scores, scores and dates, dates, diagnosed malady, and otherwise. Such database may be pre-loaded into the software, or may be dynamically updated as new data is added through research and/or clinical experience. Of course, such database may optionally reside on a centralized server, remote from the operator or clinician. Based on this collective experience, and within statistical analyses such as mean, mode, standard deviation, chi-squared, correlation and other analysis, scores may be correlated with maladies. In this way, this universe of knowledge may be used to generate a diagnosis, or at least a preliminary diagnosis or area of inquiry regarding a patient.

For example, referring to FIG. 14a, the illustrated scores on parameter A and parameter B may be indicative of low motor function. However, hypothetically if FIG. 14a were modified such that the values for parameter A were roughly the same, but for parameter B along the X axis were substantially lower, closer to zero percent or other such score, this may indicate a different diagnoses. For example, if such parameter B was correlated to memory recognition, such as optionally measured by visual recognition 400, this may lead to a different diagnoses implicating cognitive disabilities. As one hypothetical example, such diagnoses may be correlated to Alzheimer, senility, or other such parameter which are related to, but are not purely a function of physical dexterity and/or ocular dexterity; whereas the plot of FIG. 14a may be more indicative of traumatic head injury without as much loss of cognitive ability. Thus by correlating a unified diagnostic score or other score or combination of scores, the system can provide an objective, useful tool for providing or at least aiding diagnoses.

The determination of at least one diagnostic score may assist in establishing a baseline for the user, physician and/or insurance provider. Some insurance companies require progress reports to be submitted before reimbursement is provided. To that end, a user/patient's progress may optionally be calculated and reported in a patient trend output. The software and system disclosed in this application may optionally provide a consistent and reliable patient trend output, progress report and/or chart. These reports and visual illustrations of a user's progress may be valuable to health care providers as they may now be provided with clear and tangible proof of the patient/user's results.

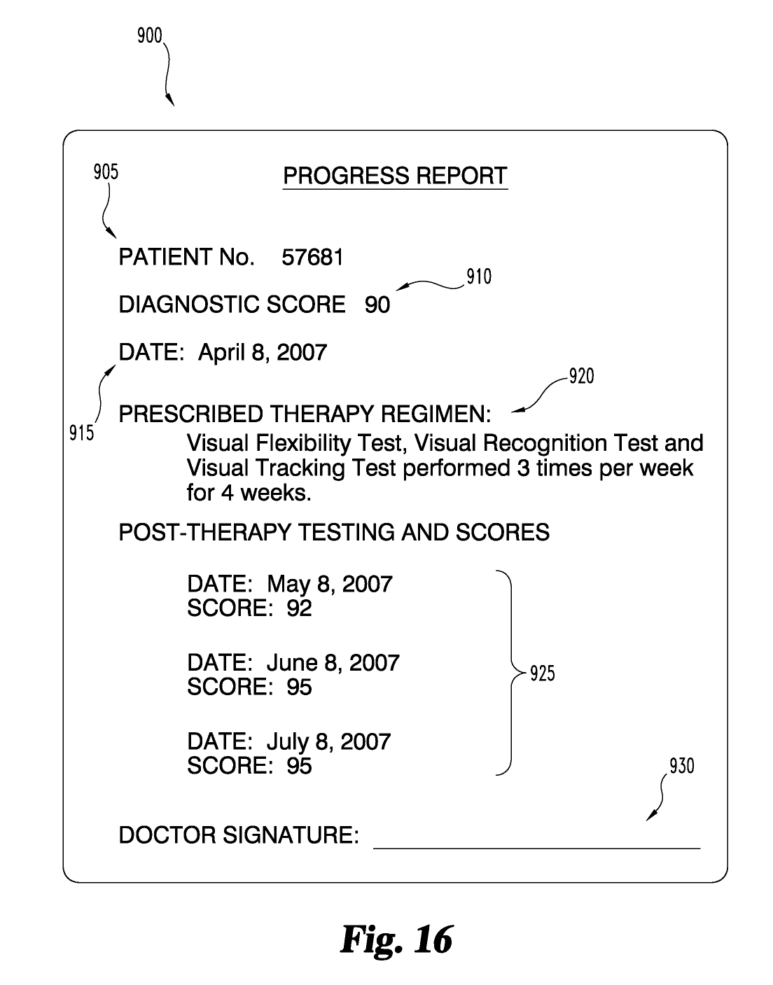

Optionally, the output from the system may be a progress report. FIG. 16 illustrates but one example of a progress report document 900. Optionally, progress report document 900 includes a patient number 905, the user's diagnostic score 910, and the date on which the diagnostic score was determined 915. The progress report document 900 may also disclose the therapy regiment 920 prescribed or instructed to the particular user. Optionally, various post-therapy scores and dates 925 are provided. These scores and dates 925 clearly depict the level of user progress over an extended period of time. A physician signature line 930 may optionally be provided in order for a physician to sign the progress report document 900 and verify the user's progress.

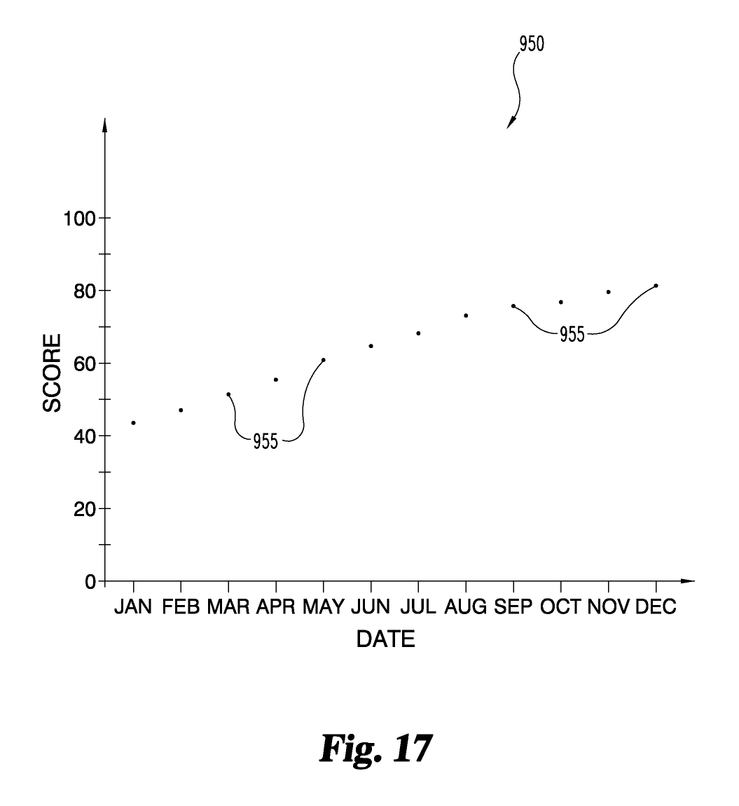

Optionally, the user's progress may be graphically illustrated. FIG. 17 illustrates progress graph 950. The horizontal axis of progress graph 950 optionally corresponds to the dates in which diagnostic and therapeutic testing was performed. The vertical axis of progress graph 950 optionally corresponds to the associated scores. As shown in FIG. 17, the various scores 955 are plotted. This graphical representation of the various diagnostic and post-therapy scores allows the user, physician, or supervising health care professional to visually ascertain the user's visual skill progress over a particular period of time.

Diagnostic evaluation and subsequent therapeutic training in accordance with the above description was conducted in the following case studies.

### Case Study #1

AG—26 y/o male sustained a traumatic brain injury from a pitched baseball in July 2005.

His initial complaints were loss of concentration and memory, difficulty reading print and comprehension, easily distracted, difficulty speaking with others over the telephone, inability to perceive pitch rotation, trajectory and speed, decrease in reaction time on the baseball field. Initial visual skills evaluation revealed reduced depth perception, convergence insufficiency, and limited visual recognition and tracking skills.

After 6 months of visual skills training on the Vizual Edge Performance Trainer platform the patient reports that in some of the initial complaint areas there is substantial improvement and some of the complaints have been completely eliminated. He has returned to pro baseball and is functioning close to the pre injury level and reports that he has regained his confidence. In the scoring spreadsheet there is pre and post training scores. His overall scores improved by 11%. Compared to normal aged ranked subjects his pre training numbers indicated he was in the 83^{rd} percentile and his post training numbers indicated he is in the 90^{th} percentile. His depth perception improved by 25%. His pre training convergence places him in the 15^{th} percentile post training 25^{th} percentile. His divergence pre training and post training percentile score is 99%. His recognition response time pre training is 45 percentile and his post training score is in the 87^{th} percentile. His response time accuracy improved by 11%. His pre training tracking response time approximately in the 1 percentile and post training percentile score is in the 75^{th} percentile. His tracking accuracy improved by 5% changing from the 65^{th} percentile and advancing to the 75^{th} percentile.

### Case Study #2

WW—37 y/o female sustained a traumatic brain injury from a snowmobile accident in January 2007. Her initial complaints were loss of concentration and memory, difficulty reading—loss of whole words on the printed materials.

Initial visual skills evaluation revealed reduced depth perception at near, convergence insufficiency, and reduced visual recognition and tracking skills.

After 10 weeks of visual skills training using the Vizual Edge Performance Trainer platform she reports reading, and concentration are at significantly improved. In the scoring spreadsheet there are pre and post training scores. Her overall scores improved by 7%. Compared to normal aged ranked subjects her pre training numbers indicated she was in the 55^{th} percentile and her post training numbers indicated she is in the 83^{rd} percentile. Her pre training convergence scores places her in the 57^{th} percentile post training 76^{th} percentile. Her divergence pre training and post training percentile score are essentially unchanged in the 70^{th} percentile. Her recognition response time pre training is in the 15^{th} percentile and her post training score is in the 77^{th} percentile. Her response time accuracy improved by 8%. Her pre training tracking response time is unchanged in the 17^{th} percentile. Her tracking accuracy improved by 8%, changing from the 5^{th} percentile and advancing to the 8^{th} percentile.

### Case Study #3

RS—46 y/o female sustained a traumatic brain injury from an automobile accident in August 2007. Her initial complaints were blurred vision, nausea, difficulty with concentration, following a target, driving a car, light sensitivity, losing her place on a page when reading, objects appear to move when stationary, difficulty drawing, work pace and quality has slowed down. Initial visual skills evaluation revealed reduced depth perception, convergence and divergence insufficiency, and very limited visual recognition and tracking skills. After 4 weeks of visual skills training using the Vizual Edge Performance Trainer platform her symptoms have improved by significantly.

In the scoring spreadsheet there are pre and post training scores. Her overall scores improved by 19%. Compared to normal aged ranked subjects her pre training numbers indicated she was in the less than 1 percentile and her post training numbers indicated she is in the 15^{th} percentile. Her depth perception improved by 25%. Her pre training convergence scores places her in the 10^{th} percentile post training 20^{th} percentile. Her divergence pre training scores placed her in the 55^{th} percentile and post training percentile score placed her in the 70^{th} percentile. Her recognition response time pre training is under the 1st percentile and her post training score is in the 1^{st} percentile. Her response time accuracy improved by 14%. Her pre training tracking response time is unchanged in the 40th percentile. Her tracking accuracy improved by 100%.

The present invention contemplates modifications as would occur to those skilled in the art. It is also contemplated that processes embodied in the present invention can be altered, rearranged, substituted, deleted, duplicated, combined, or added to other processes as would occur to those skilled in the art without departing from the spirit of the present invention. In addition, the various stages, steps, acts, procedures, techniques, phases, and operations within these processes may be altered, rearranged, substituted, deleted, duplicated, or combined as would occur to those skilled in the art. The articles “the”, “a” and “an” are not necessarily limited to mean only one, but rather are inclusive and open ended so as to include, optionally, multiple such elements.

We claim:

1. A process of diagnosing a medical patient's neurological-muscular status via an ocular interface, comprising the acts of:
(a) executing machine readable visual diagnosis software on a computer;
(b) displaying visual output from said visual diagnosis software on a visual display linked with said computer;
(c) providing an input device to permit the patient to provide input signaling to the computer in response to said visual display;
(d) conducting at least a first diagnostic test and a second, different, diagnostic test with said visual evaluation software running on said computer, said first and second diagnostic tests being from the group consisting of: visual alignment test, depth perception test, visual flexibility test, visual recognition test, and visual tracking test;
(e) calculating with said computer at least a first score from said first diagnostic test;
(f) calculating with said computer at least a second score from said second diagnostic test;
(g) calculating with said computer a unified diagnostic score based on combining at least said first score and said second score;
(h) outputting said unified diagnostic score in a first output.

2. The process of claim 1, and further comprising the act of patient therapy, said therapy comprising the acts of the patient performing at least a first therapy regimen with said visual evaluation software running on said computer, said first therapy regimen being from the group consisting of: visual flexibility test, visual recognition test, and visual tracking test.

3. The process of claim 1 wherein said first output comprises a physician's prescription document which includes at least: (i) patient identification; and, (ii) a therapy prescription.

4. The process of claim 1, wherein said computer stores in computer memory associated with said patient the results of said first therapy regimen, and wherein said computer outputs said results in a patient trend output.

5. The process of claim 1, wherein at least one of said diagnostic tests measures the time between an image being displayed to the patient on said display and the patient's response thereto via said input device.

6. The process of claim 1, wherein at least one of said diagnostic tests measures the accuracy between the patient's response and the image displayed to the patient.

7. The process of claim 1, wherein at least one of the said diagnostic tests measures the patient's memory by temporarily displaying a memory image to the patient on said display and then removing that memory image after an amount of time has lapsed, the patient responding via said input device after said lapse to replicate said memory image.

8. The process of claim 1, wherein at least one of said diagnostic tests comprises the acts of covering the patient's left eye with a lens having a first color, and covering the patient's right eye with a lens having a second, different color, and wherein said diagnostic tests display on said display at least a first image in said first color and at least a second image in said second color.

9. The process of claim 1, wherein at least one of said diagnostic tests comprises the acts of conducting at least a third, different, diagnostic test with said visual evaluation software running on said computer, said third diagnostic tests being from the group consisting of: visual alignment test, depth perception test, visual flexibility test, visual recognition test, and visual tracking test;
calculating with said computer at least a third score from said third diagnostic test;
calculating with said computer a unified diagnostic score based on combining at least said first, second and third score.

10. The process of claim 9, wherein at least one of said diagnostic tests comprises the acts of conducting at least a fourth, different, diagnostic test and a fifth, different diagnostic test with said visual evaluation software running on said computer, said fourth and fifth diagnostic tests being from the group consisting of: visual alignment test, depth perception test, visual flexibility test, visual recognition test, and visual tracking test;
calculating with said computer at least a fourth score from said fourth diagnostic test;
calculating with said computer at least a fifth score from said fifth diagnostic test;
calculating with said computer a unified diagnostic score based on combining at least said first, second, third, fourth and fifth score.

11. The process of claim 1, wherein said scores are numeric, and wherein at least one coefficient is multiplied by at least one of said scores as part of calculating said unified score.

12. The process of claim 1, wherein said output includes a graphical representation of the patient's diagnostic testing, wherein the graphical representation shows at least two parameters plotted along two respective dimensions.

13. The process of claim 2 wherein said first output comprises a physician's prescription document which includes at least: (a) patient identification; and, (b) a therapy prescription.

14. The process of claim 13, wherein said computer stores in computer memory associated with said patient the results of said first therapy regimen, and wherein said computer outputs said results in a patient trend output.

15. The process of claim 14, wherein at least one of said diagnostic tests measures the time between an image being displayed to the patient on said display and the patient's response thereto via said input device.

16. The process of claim 15, wherein at least one of said diagnostic tests measures the accuracy between the patient's response and the image displayed to the patient.

17. The process of claim 16, wherein at least one of the said diagnostic tests measures the patient's memory by temporarily displaying a memory image to the patient on said display and then removing that memory image after an amount of time has lapsed, the patient responding via said input device after said lapse to replicate said memory image.

18. The process of claim 17, wherein at least one of said diagnostic tests comprises the acts of covering the patient's left eye with a lens having a first color, and covering the patient's right eye with a lens having a second, different color, and wherein said diagnostic tests display on said display at least a first image in said first color and at least a second image in said second color.

19. The process of claim 18, wherein at least one of said diagnostic tests comprises the acts of conducting at least a third, different, diagnostic test with said visual evaluation software running on said computer, said third diagnostic tests being from the group consisting of: visual alignment test, depth perception test, visual flexibility test, visual recognition test, and visual tracking test;
calculating with said computer at least a third score from said third diagnostic test;
calculating with said computer a unified diagnostic score based on combining at least said first, second and third score.

20. The process of claim 19, wherein at least one of said diagnostic tests comprises the acts of conducting at least a fourth, different, diagnostic test and a fifth, different diagnostic test with said visual evaluation software running on said computer, said fourth and fifth diagnostic tests being from the group consisting of: visual alignment test, depth perception test, visual flexibility test, visual recognition test, and visual tracking test;
calculating with said computer at least a fourth score from said fourth diagnostic test;
calculating with said computer at least a fifth score from said fifth diagnostic test;
calculating with said computer a unified diagnostic score based on combining at least said first, second, third, fourth and fifth score.

21. The process of claim 20, wherein said scores are numeric, and wherein at least one coefficient is multiplied by at least one of said scores as part of calculating said unified score.

22. The process of claim 21, wherein said output includes a graphical representation of the patient's diagnostic testing, wherein the graphical representation shows at least two parameters plotted along two respective dimensions.

23. The process of claim 5, wherein at least one of said diagnostic tests measures the accuracy between the patient's response and the image displayed to the patient.

24. The process of claim 5, wherein at least one of said diagnostic tests comprises the acts of covering the patient's left eye with a lens having a first color, and covering the patient's right eye with a lens having a second, different color, and wherein said diagnostic tests display on said display at least a first image in said first color and at least a second image in said second color.

25. The process of claim 8, wherein at least one of the said diagnostic tests measures the patient's memory.

26. The process of claim 25, wherein said scores are numeric, and wherein at least one coefficient is multiplied by at least one of said scores as part of calculating said unified score.

27. The process of claim 25, wherein said output includes a first parameter correlated to said memory test, and wherein said output further includes a second parameter correlated to testing using said first and second colored lenses.

28. The process of claim 1, wherein at least one of said diagnostic tests measures the time between an image being displayed to the patient on said display and the patient's response thereto via said input device; and, wherein at least one of said diagnostic tests measures the accuracy between the patient's response and the image displayed to the patient; and, wherein said output includes a first parameter correlated to said time measurement, and wherein said output further includes a second parameter correlated to accuracy measurement.

29. The process of claim 1 wherein said input device may be activated by the patient without requiring physical movement of the patients arms or legs.

30. The process of claim 1 and further comprising the act of comparing at least one of said scores with statistically compiled score values in a computer database, said database including data of diagnoses correlations between visual skills testing and medical indications from a patient population, and generating an output diagnosis for the present patient based on said patient's scoring.

31. The process of claim 27 and further comprising the act of comparing at least one of said scores with statistically compiled score values in a computer database, said database including data of diagnoses correlations between visual skills testing and medical indications from a patient population, and generating an output diagnosis for the present patient based on said patient's scoring.

32. The process of claim 1 wherein said input device is hand-held and may be activated by the patient with their hand digits and without requiring physical movement of the patients arms or legs.

33. The process of claim 3 wherein said input device may be activated by the patient without requiring physical movement of the patients arms or legs.

34. The process of claim 5, wherein at least one of said diagnostic tests comprises the acts of covering the patient's left eye with a lens having a first polarity, and covering the patient's right eye with a lens having a second, different polarity, and wherein said diagnostic tests display on said display at least a first image in said first polarity and at least a second image in said second polarity.

35. A process of diagnosing a medical patient's neurological-muscular status via an ocular interface, comprising the acts of:
(a) executing machine readable visual diagnosis software on a computer;
(b) displaying visual output from said visual diagnosis software on a visual display linked with said computer;
(c) providing an input device to permit the patient to provide input signaling to the computer in response to said visual display;
(d) conducting at least a first diagnostic test and a second, different, diagnostic test with said visual evaluation software running on said computer;
(e) calculating with said computer at least a first score from said first diagnostic test; and, (f) outputting in a first output comprising a physician's prescription document which includes at least: (i) patient identification; and, (ii) a therapy prescription.

36. The process of claim 35, wherein said diagnostic test measures the time between an image being displayed to the patient on said display and the patient's response thereto via said input device.

37. The process of claim 35, wherein said diagnostic test measures the accuracy between the patient's response and the image displayed to the patient.

38. The process of claim 35, wherein said diagnostic test measures the patient's memory by temporarily displaying a memory image to the patient on said display and then removing that memory image after an amount of time has lapsed, the patient responding via said input device after said lapse to replicate said memory image.

39. The process of claim 35, wherein said diagnostic test comprises the acts of covering the patient's left eye with a lens having a first color, and covering the patient's right eye with a lens having a second, different color, and wherein said diagnostic tests display on said display at least a first image in said first color and at least a second image in said second color.

40. The process of claim 35, wherein said input device may be activated by the patient without requiring physical movement of the patients arms or legs.

41. The process of claim 35, wherein at least one of said diagnostic tests comprises the acts of covering the patient's left eye with a lens having a first polarity, and covering the patient's right eye with a lens having a second, different polarity, and wherein said diagnostic tests display on said display at least a first image in said first polarity and at least a second image in said second polarity.

42. A process of tracking a medical patient's neurological-muscular status via an ocular interface, comprising the acts of:
(a) executing machine readable visual diagnosis software on a computer;
(b) displaying visual output from said visual diagnosis software on a visual display linked with said computer;
(c) providing an input device to permit the patient to provide input signaling to the computer in response to said visual display;
(d) conducting at least a first diagnostic test with said visual evaluation software running on said computer;
(e) calculating with said computer at least a first score from said first diagnostic test;
(f) storing said at least first score and the results of subsequent, similar diagnostic tests into computer memory; and,
(h) outputting a patient trend output of said scores.

43. The process of claim 42, wherein said computer memory is maintained remotely from said computer.

44. The process of claim 42, wherein the patient must login to a web-site by entering the appropriate identification information before said visual diagnosis software may be used.

45. The process of claim 42, wherein said input device may be activated by the patient without requiring physical movement of the patients arms or legs.

46. A process of tracking a medical patient's neurological-muscular status via an ocular interface, comprising the acts of:
(a) executing machine readable visual diagnosis software on a computer;
(b) displaying visual output from said visual diagnosis software on a visual display linked with said computer;
(c) providing an input device to permit the patient to provide input signaling to the computer in response to said visual display;
(d) conducting at least a first diagnostic test;
(e) calculating with said computer at least a first score from said first diagnostic test;
(f) comparing at least one of said scores with statistically compiled score values in a computer database, said database including data of diagnoses correlations between visual skills testing and medical indications from a patient population; and,
(g) outputting a diagnosis for the present patient based on said patient's scoring.

47. A system for diagnosing a medical patient's neurological-muscular status via an ocular interface, comprising:
(a) means for executing machine readable visual diagnosis software on a computer;
(b) a visual output linked with said computer;
(c) an input device to permit the patient to provide input signaling to the computer in response to said visual display;
(d) means for conducting at least a first diagnostic test and a second, different, diagnostic test with said visual evaluation software running on said computer, said first and second diagnostic tests being from the group consisting of: visual alignment test, depth perception test, visual flexibility test, visual recognition test, and visual tracking test;
(e) means for calculating with said computer at least a first score from said first diagnostic test;
(f) means for calculating with said computer at least a second score from said second diagnostic test;
(g) means for calculating with said computer a unified diagnostic score based on combining at least said first score and said second score;
(h) means for outputting said unified diagnostic score in a first output.
